(19) United States
(12) Patent Application Publication
(10) Pub. No.: US 2010/0144610 A1
(43) Pub. Date: Jun. 10, 2010

(54) PHARMACEUTICAL FORMULATIONS

(75) Inventors: Saeed M. Chaudhry, Clifton, NJ (US); Julianne Berry, Westfield, NJ (US); Joel A. Sequeira, Venice, FL (US)

Correspondence Address: MERCK;PATENT DEPARTMENT (K-6-1, 1990), 2000 GALLOPING HILL ROAD, KENILWORTH, NJ, 07033-0530, US

(73) Assignee: Schering Corporation

(21) Appl. No.: 12/707,424

(22) Filed: Feb. 17, 2010

Related U.S. Application Data
(63) Continuation of application No. 11/672,400, filed on Feb. 7, 2007, now abandoned.
(60) Provisional application No. 60/771,921, filed on Feb. 9, 2006.

(51) Int. Cl. A61K 31/4245; A61K 31/56; A61K 31/454; A61K 31/4545; A61K 31/55; A61K 31/495; A61K 31/60; A61K 31/53; A61K 31/542; A61K 31/439; A61K 31/551; A61K 31/545; A61K 31/65; A61K 38/12; A61K 31/43; A61P 11/00

(52) U.S. Cl. 514/11; 514/364; 514/171; 514/322; 514/318; 514/217.05; 514/255.04; 514/161; 514/243; 514/224.5; 514/291; 514/220; 514/200; 514/152; 514/192

(57) ABSTRACT

An aspect of the present invention provides for a medicament including a solution containing pleconaril or a pharmaceutically acceptable salt thereof, wherein at least one solvent including the solution is a pleconaril-dissolving hydrofluorocarbon.

This application claims priority to Provisional Application No. 60/771,921 filed Feb. 9, 2006.

### FIELD OF INVENTION

The present invention is directed to formulations containing Pleconaril either alone or in combination with one or more other pharmaceutically active agents in novel dosage forms and methods of using the same.

### BACKGROUND OF THE INVENTION

Identification of any reference in this section or any section of this application is not an admission that such reference is prior art to the present invention. Pleconaril is known as 1,2,4-oxadiazole3-[3,5-Dimethyl-4-[3-(3-methyl-5-isoxazolyl)propoxy]phenyl]-5-(trifluoremethyl). It has other names such as PICOVIR®, VP 63843 and Win 63843. Pleconaril is a picornavirus replication inhibitor and is a new chemical entity (NCE) which has been shown to be active against rhinoviruses. According to the Merck Index, pleconaril may be prepared in accordance with U.S. Pat. No. 5,464,848, which is incorporated by reference.

Due to the efficacy of Pleconaril as an anti-viral agent for the treatment of the common cold, it would be beneficial to administer it along with other medications and/or in certain dosage forms that relieve symptoms associated with the common cold, viral induced respiratory diseases and/or other disease states. NCE drugs may raise safety issues when administered systemically. Accordingly, in administering pleconaril, for example in combating rhinovirus infections, it is preferred to administer this class of drugs topically, for example, by respiratory inhalation, for example, inhalation through the mouth (oral inhalation delivery) for treatment of the upper and/or lower airways and inhalation through the nose (nasal inhalation delivery) for treatment of the sinus and nasal mucosa.

Medicaments directed at respiratory delivery in general may comprise a liquid carrier, for example, aqueous based or lipid based, and include both suspensions of the therapeutic agent in a carrier and solutions having the therapeutic agent dissolved in the carrier. Heretofore, in general, medicaments formulated for nasal inhalation have been aqueous based, either aqueous suspensions of insoluble therapeutic agents or aqueous solutions of soluble therapeutic agents. Some therapeutic agents have been formulated as a dry particulate suitable for administration by oral inhalation.

The dosing consistency and efficacy of medicaments in the form of dry powder and suspensions for respiratory delivery depends upon the constituent particles having a small mean particle size and a narrow particle size distribution. This has been discussed, for example, see Pritchard, J. N., The Influence of Lung Deposition on Clinical Response, Journal of Medicine, 2001, 14(1). pp. 19 to 26, and Meyer, K. C. et al., Drug Delivery to the Lung in Polymeric Site-Specific Pharmacology; Eds, A. J. Domb; John Wiley and Sons: New York, 1994, ppp 347-367. Additionally, effective topical treatment of a condition with particulate material is limited by the ability of the therapeutic compound contained in a powder or suspension to be dispersed effectively across the site of treatment. Accordingly, conditions which alter or affect mean particle size and/or particle size distribution in a suspension or dry powder medicament can affect both the ability of the therapeutic agent in the medicament to be dispersed at the intended site of treatment and its bioavailability once administered. Compositions comprising a suspension are subject to physical instability by flocculation and/or aggregation. Dry powder compositions are subject to aggregation during storage. In addition, topical application of particulate materials is limited in its ability to disperse the therapeutic agent over the site of application. This limitation makes treatment of some conditions by topical application of a particulate therapeutic agent impractical. Moreover, in some cases it is more efficient and effective to supply multiple therapeutic agents to a treatment site in the management of a disease state which may have multiple symptoms, each of which is responsive to a different therapeutic agent. U.S. application Ser. No. 11/196,745, filed Aug. 3, 2005, which is incorporated herein by reference in its entirety, discusses combinations of pleconaril with a variety of therapeutic agents although it does not discuss or suggest dosage forms either containing pleconaril solutions or containing suspensions of pleconaril in a thixotropic carrier.

Pleconaril is insoluble in aqueous solvents and for this reason has been prepared as an aqueous particulate suspension containing solely pleconaril as a therapeutic agent. Heretofore, when these suspensions have been administered by nasal inhalation they have lacked the ability to be retained in the nasal cavity.

### Objectives

In view of the foregoing, what is needed is a medicament comprising a solution containing pleconaril, and optionally comprising one or more additional therapeutic agents. What is needed also is a medicament comprising a solution containing pleconaril that can be delivered in the form of an aerosol, for example, via a metered dose inhaler, or by a metered pump spray for inhalation delivery.

What is needed also is an aqueous suspension of pleconaril, optionally comprising one or more additional therapeutic agents, which suspension has thixotropic behavior suitable for administration by nasal inhalation and sufficient viscosity after administration to be retained in the nasal cavity. These and other objectives and/or advantages are provided by the present invention.

### SUMMARY OF THE INVENTION

Accordingly, in one aspect of the present invention there is disclosed a medicament comprising a solution containing pleconaril or a pharmaceutically acceptable salt thereof, said solution comprising at least one solvent selected from the group consisting of pleconaril-dissolving glyceride oils, pleconaril-dissolving hydrofluorocarbons, and mixtures of two or more thereof.

In some embodiments the solution containing pleconaril comprises one or more solvents selected from the group consisting of triesters which can be made by esterifying a mixture of capric and caprylic acid with glycerine. In some embodiments the solution containing pleconaril comprises at least one member of the group consisting of 1,1,1,2,3,3,3 heptafluoro propane, 1,1,1,2 tetrafluoro ethane, and mixtures thereof. In some embodiments the solution containing pleconaril comprises Miglyol 812® (a triglyceride made from a mixture of saturated fatty acids comprising from about 50 wt. % to about 65 wt % C₈ and from about 30 wt. % to about 45 wt. % C₁₀ from Sasol North America Inc.).

Another aspect of the present invention is the provision of a medicament comprising: (i) at least one solution containing pleconaril or a pharmaceutically acceptable salt thereof; and (ii) one or more members of the group consisting of corticosteroids, antihistamines, expectorants, non-steroidal anti-inflammatory agents, decongestants, anti-cholinergics, pharmaceutically acceptable zinc salts, antibiotics, histamine H₃ receptor antagonists, leukotriene D₄ antagonists, leukotriene inhibitors, P₂Y agonists, syk kinase analogues, echinaceia, vitamin C, and vitamin E.

Another aspect of the invention is the provision of a medicament comprising a solution containing pleconaril or a pharmaceutically acceptable salt thereof, and optionally one or more additional therapeutic agents, wherein the solution is adapted to be administered via an inhalation route. In some preferred embodiments, the medicament comprises a 1,1,1,2,3,3,3 heptafluoro propane solution containing pleconaril, or a pharmaceutically acceptable salt thereof, and optionally, associated therewith, mometasone furoate, and optionally suspended therein, oxymetazoline hydrochloride. In some embodiments, the medicament comprises an aqueous solution of oxymetazoline HCl emulsified with a solution containing pleconaril or a pharmaceutically acceptable salt thereof.

In some embodiments of the present invention the medicament comprising a solution containing pleconaril (referred to herein also as a “pleconaril medicament”) is contained by itself in a device for administration of the pleconaril medicament. In some embodiments, a pleconaril medicament and one or more separate medicaments containing one or more additional therapeutic agents are packaged together in a device for administering the pleconaril medicament along with one or more separate medicaments comprising one or more members of the group consisting of corticosteroids, antihistamines, expectorants, non-steroidal anti-inflammatory agents, decongestants, anti-cholinergics, pharmaceutically acceptable zinc salts, antibiotics, histamine H₃ receptor antagonists, leukotriene D₄ antagonists, leukotriene inhibitors, P₂Y agonists, syk kinase analogues, echinaceia, vitamin C, and vitamin E, wherein the device is adapted for simultaneous, sequential or separate administration of the pleconaril medicament and the one or more separate medicaments copackaged with it. In some embodiments at least one pleconaril medicament is packaged in kit form, optionally along with one or more separate medicaments containing one or more additional therapeutic agents to be simultaneously, sequentially or separately administered in conjunction with administration of the pleconaril medicament, and including a device facilitating inhalation administration of the pleconaril medicament included in the kit.

In some embodiments the pleconaril medicament which optionally contains one or more additional therapeutic agents, is administered, either alone or in conjunction with one or more separate medicaments containing additional therapeutic agents, in the treatment of an upper or lower respiratory, viral, inflammatory or obstructive airways disease to a patient in need of such treatment.

In some embodiments, the medicament comprising a solution containing pleconaril is administered via an inhalation route selected from oral inhalation and nasal inhalation. In some preferred embodiments, administration is accomplished utilizing a device selected from a nebulizer, a metered pump-spray device, and a pressurized metered dosing inhaler. In some embodiments, utilizing an inhalation device for administering a medicament comprising a solution containing pleconaril, the inhalation device, optionally, may be adapted by the administrator for administration of the medicament through either an oral or nasal inhalation route. In one embodiment, a single pressurized metered dose inhaler may be adapted for oral inhalation or nasal inhalation routes simply by switching between an actuator that is designed for nasal delivery and an actuator designed for oral delivery. In some embodiments, a medicament comprising a solution containing pleconaril is provided in a form for topical application, for example to the dermis.

In another aspect, the present invention provides a medicament comprising an aqueous suspension of pleconaril, and optionally one or more additional therapeutic agents, formulated for delivery by a metered dose pump spray device for administration to nasal mucosa. In some embodiments, the pleconaril is co-suspended with one or more additional water insoluble therapeutic agents, for example, mometasone furoate, and optionally contains also one or more additional water soluble therapeutic agents, for example, oxymetazoline HCl. In some embodiments, the medicament suspension comprises a thixotropic carrier solution which has sufficient viscosity after administration to provide “no-drip” characteristics when applied to nasal mucosa.

In some preferred embodiments, the medicament comprising an aqueous suspension of pleconaril is a nasal spray composition comprising water, pleconaril, optionally oxymetazoline or a pharmaceutically acceptable salt thereof, about 2.5 to about 3.5 weight percent of a mixture of microcrystalline cellulose and an alkali metal carboxyalkylcellulose, and about 0.5 to about 5 weight percent of polyvinylpyrrolidone, wherein complex viscosity of the composition increases to at least about 10 times a minimum complex viscosity of the composition as measured under high shear conditions, within about 20 seconds after the high shear conditions terminate.

Another aspect of the present invention is the provision of opthalmic compositions. Preferred ophthalmic compositions comprise a liquid, an ointment, or an aqueous gel. In one preferred embodiment, the composition is a water-in-oil emulsion with the additional therapeutic agent(s) dissolved or suspended within aqueous droplets which are in turn suspended in a lotion or flowable ointment base comprising, e.g., petrolatum, mineral oil, and the like, wherein the composition includes pleconaril dissolved in a suitable pleconaril-dissolving glyceride oil or a suitable pleconaril-dissolving HFC.

In some embodiments, a medicament comprising a solution containing pleconaril is provided in a liquid oral dosage form. In some embodiments, a medicament comprising a solution containing pleconaril is provided encapsulated in a gelatin capsule.

Other advantages of the present invention will be apparent to those of skill in the art.

### DETAILED DESCRIPTION OF THE INVENTION

There follows the definition of terms used in the description of the present invention.

The term “pharmaceutically acceptable salt” refers to a non-toxic salt prepared from pharmaceutically acceptable acids or bases including inorganic acids, inorganic bases, organic acids, and organic bases. Examples of suitable inorganic acids are hydrochloric, hydrobromic, hydroiodic, sulfuric, and phosphoric acid. Appropriate organic acids may be selected, for example, from aliphatic, aromatic, carboxylic and sulfonic classes of organic acids, examples of which are formic, acetic, propionic, succinic, glycolic, glucuronic, maleic, furoic, glutamic, benzoic, anthranilic, salicylic, phenylacetic, mandelic, embonic (pamoic), methanesulfonic, ethanesulfonic, pantothenic, benzenesulfonic, stearic, sulfanilic, algenic, and galacturonic acid. Examples of suitable inorganic bases include metallic salts made from aluminum, calcium, lithium, magnesium, potassium, sodium, and zinc. Appropriate organic bases may be selected, for example, from N,N-dibenzylethylenediamine, chloroprocaine, choline, diethanolamine, ethylenediamine, meglumaine (N-methylgulcaine), lysine and procaine.

The phrase “therapeutically effective amount” means that amount of a medicament which when administered supplies an amount of one or more pharmaceutically active agents contained therein to provide a therapeutic benefit in the treatment or management of a disease or disease state.

Dosage form—refers to the administrable form of a medicament composition provided in a measured or unit amount, and includes at least one therapeutic agent in association with one or more other excipients comprising a delivery system, for example, a carrier, a diluent, and a coloring agent. Examples of dosage forms include, a capsule, a measured amount of aerosol presented for inhalation, and a measured amount of liquid presented for imbibing.

Heretofore, pleconaril was known to be soluble only in liquids of low polarity which were not suitable for forming an aerosol, for example, corn oil and ethanol. Accordingly, a medicament containing pleconaril suitable for administration via an inhalation route heretofore has relied upon providing pleconaril in a particulate form for inhalation administration. Examples of this include a suspension of pleconaril as the sole therapeutic agent in a liquid carrier, generally an aqueous carrier, which is dispersed as an aerosol, and entraining a pleconaril-containing powdered inhalant in an air stream, each of which is administered by inhalation. However, in some circumstances, delivery of a particulate form of pleconaril is disadvantageous, for example in the treatment of piconovirus induced illness, for example, the common cold, wherein inhalation of particulate pleconaril applies the powder to the affected tissue, but the particulate nature of the medicament leaves areas of the tissue deprived of a therapeutic level of pleconaril. In the management of a disease state or the provision of a therapy, for example, in the treatment of colds, complete coverage of the tissue to be treated with the therapeutic agent is advantageous.

Surprisingly, the inventors have discovered that pleconaril can be dissolved in certain glyceride oils, providing a medicament comprising a solution containing pleconaril that is suitable for dispersion as an aerosol delivered from a pump spray bottle. Advantageously, medicaments comprising a solution containing pleconaril of this type can be administered utilizing, for example, a metered pump spray dispenser, a metered, pressurized aerosol inhaler (when packaged with a propellant), or utilized in a nebulizer. Of further advantage, certain glyceride oils, for example, Miglyol 812® (a triglyceride made from a mixture of saturated fatty acids comprising from about 50 wt. % to about 65 wt. % C₈ and from about 30 wt. % to about 45 wt % C₁₀ from Sasol North America Inc.) are miscible with hydrofluorcarbon propellants commonly used in MDI devices. Medicaments comprising a solution containing pleconaril are suitable for inhalation administration to a patient having a condition treatable by topical application of pleconaril. For convenience, the glyceride oils comprising these solutions (described in detail below) are referred to also herein as “pleconaril-dissolving glyceride oils”. It is contemplated that pleconaril-containing solutions utilizing pleconaril-dissolving glyceride oils will find utility in the preparation of medicaments for delivery by oral ingestion, inhalation (nasal and oral), and topical application to the external skin and eyes. It is believed that medicaments comprising pleconaril-dissolving glyceride oil solutions of pleconaril will find their greatest utility in administration by oral inhalation from a nebulizer and nasal and oral inhalation of an aerosol of the medicament provided by a metered pump spray or delivered as an aerosol from a pressurized metered inhaler device and in topically applied ophthalmic formulations.

Suitable pleconaril-dissolving glyceride oils have a room temperature dynamic viscosity of less than about 33 cP and include triglycerides made by esterifying glycerine in the presence of capric acid, caprylic acid, and mixtures of capric and caprylic acid. Preferably, pleconaril-dissolving glyceride oils are selected from those comprising triglycerides produced by esterification of glycerine in the presence of a mixture of caprylic acid and capric acid and which are generally recognized as safe for human contact. More preferred are triglycerides produced by esterification of glycerine in the presence of a mixture of capric and caprylic acid comprising up to about 45 wt. % capric acid, with the remainder of the fatty acid mixture substantially comprising caprylic acid. Preferred are triglycerides produced by esterfying glycerine in the presence of a mixture of fatty acids comprising from about 20 wt. % to about 45 wt. % capric acid and from about 50 wt. % to about 80 wt. % caprylic acid with no more than a total of 5 wt. % of the fatty acid mixture comprising a combination of C₆, C₁₂ and C₁₄ fatty acids. More preferred are triglycerides produced by esterfying glycerine in the presence of a mixture of fatty acids comprising from about 30 wt. % to about 45 wt. % capric acid and from about 50 wt. % to about 65 wt. % caprylic acid with no more than a total of 5 wt. % of the fatty acid mixture comprising a combination of C₆, C₁₂ and C₁₄ fatty acids Examples of suitable glyceride oils comprising glycerine esterified in the presence of a mixture of capric and caprylic acid that are available commercially include, but are not limited to, Miglyol 812® available from Sasol North America. Sasol's product literature describes Miglyol 812® as a triglyceride made from a mixture of fatty acids comprising from about 50 wt. % to about 65 wt. % caprylic acid (herein, C₈) and from about 30 wt % to about 45 wt. % capric fatty acid (herein C₁₀), with no more than 2 wt % caproic acid, 2 wt % lauric acid and 1 wt % linoleic acid present in the mixture, described herein for convenience as “a triglyceride made from a mixture of saturated fatty acids comprising from about 50 wt. % to about 65 wt. % C₈ and from about 30 wt. % to about 45 wt. % C₁₀ from Sasol North America Inc.”

Additionally, the inventors have surprisingly discovered that pleconaril can be dissolved in certain condensed phases of hydrofluorocarbons (herein for convenience referred to also as “pleconaril-dissolving hydrofluorocarbons”). Accordingly, there is provided a medicament comprising a pleconaril-containing solution that is suitable for administration from a pressurized metered dose inhaler device. If a hydrofluorocarbon (HFC) is selected from which to prepare a pleconaril-containing solution that has a room temperature vapor pressure that is sufficiently high, the selected HFC can act both as a solvent and as a propellant. An example of one such HFC is 1,1,1,2,3,3,3 heptafluoropropane (HFA 227, Solvay), which has a room temperature vapor pressure of approximately 66 psia. It will be appreciated that by selecting an HFC having a low room temperature vapor pressure that is sufficiently low that it does not boil at room temperature will afford HFC solutions of pleconaril which are suitable for administration utilizing a metered pump spray device or a nebulizer. It will be appreciated that such low vapor pressure HFC solutions can also be administered from a pressurized metered dose inhaler device if a suitable propellant is packaged along with the solution.

It is believed that medicaments comprising pleconaril-dissolving HFC solutions of pleconaril will find their greatest utility in administration by inhalation, either via nasal and oral inhalation, of an aerosol of the medicament provided by a metered pump spray or delivered from a pressurized metered inhaler device.

Preferred pleconaril-dissolving hydrofluorocarbons are those in which pleconaril, or a pharmaceutically acceptable salt thereof, exhibits a solubility at ambient temperature (about 25° C.) of at least about 1 g/ml, and which have an ambient temperature (about 25° C.) vapor pressure of from about 66 psia, for example 1,1,1,2 tetrafluoroethane, for example HFA-134a (DuPont), to about 96 psia, for example 1,1,1,2,3,3,3 heptafluoropropane, for example HFA-227 (Solvay).

Because it is preferred to deliver pleconaril topically rather than systemically, it is believed that HFC-based medicaments will find their broadest utility in medicament compositions which are administered either by oral inhalation or nasal inhalation. For applications wherein the medicament is to be administered orally to the gastrointestinal tract as a liquid, it is preferred to utilize pleconaril-dissolving glyceride oils, although it will be appreciated that sufficiently non-volatile HFC's may also be employed.

In one mode of the present invention, a medicament comprising a pleconaril containing-solution is provided by dissolving a weighed amount of pleconaril in a pleconaril-dissolving glyceride oil or in a pleconaril-dissolving hydrofluorocarbon solvent to provide a solution containing pleconaril. A medicament is prepared by combining with an appropriate amount of the solution containing pleconaril, optionally, one or more other desired therapeutic agents and optionally one or more other excipients, for example, a surfactant to promote desired aerosol droplet formation, and charging the resultant solution containing pleconaril into the desired administration apparatus, for example, a metered pump spray dispenser, a pressurized metered dose inhaler (along with a propellant if needed), and a nebulizer.

In one mode of the present invention, a medicament comprising a solution containing pleconaril is provided by placing a weighed amount of pleconaril into a suitable vessel, for example, a pressurized metered dose inhaler body, applying a metering valve onto the body, and filling a calculated weight of a solvent selected from a pleconaril-dissolving glyceride oil, a pleconaril-dissolving hydrofluorocarbon, and mixtures of two or more thereof into the vessel along with additional propellant if needed.

It will be appreciated that inhalation delivery of a medicament requires the provision of an aerosol of the medicament comprising droplets of a suitable size to administer the medicament to the intended location within the nasal or respiratory tract. Investigators have reported the results of studies of effective inhalation administration of aerosols, for example, Newman, S. P. Aerosol Generators and Delivery Systems, Respiratory Care, 1991, 36, pp. 939-951, Clay, M. et al., Effect of Nebulized Aerosol Size on Lung Deposition in Patients With Mild Asthma, Thorax 1987, 42, 120, Dolovich, M. B. et al., Optimal Delivery of Aerosols from Metered Dose Inhalers, Chest, 80 (supplemental) 1981, pp. 911-915, Pritchard, J. N., The Influence of Lung Deposition on Clinical Response, Journal of Medicine, 2001, 14(1), S19-S26 (2001), and Meyer, K. C. et al., Drug Delivery to the Lung in Polymeric Site-Specific Pharmacology; Eds, A. J. Domb; John Wiley and Sons: New York, 1994, ppp 347-367, each of which is incorporated in its entirety by reference. Accordingly, medicaments of the invention for use in these delivery devices may optionally contain a surfactant, as will be appreciated by those of skill in the art, which aids in the provision of droplets having a narrow size range and of a suitable average size to form a dispersion appropriate to administer the medicament to the intended site of administration. For nasal administration, it is preferred for the dispersion to comprise droplets having a average diameter [D(v, 0.5)] of from about 20 microns to about 100 microns, and wherein 90% of the droplets [D(v, 0.9)] have a diameter of not more than 200 microns, 10% of the droplets [D(v, 0.1)] have a diameter of not more than 45 microns. For administration via oral inhalation, the mass median aerodynamic particle size should be from about 1 micron to about 5 microns.

Discussed next are examples of various delivery devices which may be used to administer the medicaments of the present invention via inhalation, and thus administer a medicament comprising a solution containing pleconaril topically rather than systemically. These include metered pump spray dispensers, pressurized metered dose inhalers, and nebulizers. Metered pump spray dispensers comprise a pump which is manually operated that when actuated pumps a measured amount of a medicament contained therein through an orifice in the provision of an aerosol of droplets having a respirable size of appropriate average diameter and size distribution to reach the site of action to which the medicament is to be administered upon inhalation of the aerosol. An example of one such manually actuated pump which is suitable for providing an aerosol of the inventive compositions described herein is the VP3 line of pumps available from Valois Pharmaceutical Division, France, for example a VP3/93 model which is a crimp-on 93 microliter manually operated metered dose aerosol pump. Examples of pump spray dispensers suitable for use with medicament formulations of the present invention include, but are not limited to, pump spray bottles which administer specific, measured amounts of liquid or suspensions, for example, those used to dispense an aqueous suspension commercially available under the trade name NASONEX® Nasal Spray and the spray bottle disclosed in the Schering Corporation industrial Design Deposit DM/026304, registered by the Hague Union on Jun. 1, 1993 (each are available from Schering Corporation).

Pressurized metered-dose inhalers (“MDI”) contain propellants, for example, chlorofluorocarbon propellants, for example, CFC-11, CFC-12, hydrofluorocarbon propellants, for example, HFC-134A, HFC-227, to produce a precise quantity of an aerosol of the medicament contained with the device, which is administered by inhaling the aerosol either orally (entering either the upper or lower respiratory tract), or nasally, treating the nasal mucosa and/or the sinus cavities.

Examples of pressurized metered dose inhalers which may be used to deliver medicament formulations of the present invention include, but are not limited to the MDI device currently on the market for delivery of Proventil HFA, available from Schering Plough.

In some embodiments utilizing either of a pressurized metered dose inhaler, or a metered pump spray aerosol delivery device containing a medicament formulation of the present invention, the delivery device may comprise two interchangeable actuators, one each for oral and nasal inhalation delivery of the medicament. Thus, there is provided a mechanism for delivering the medicament to treat both the oral and nasal sites of viral infection. A typical actuator for nasal delivery may be circular with an orifice diameter of about one millimeter. An actuator for use in oral delivery can be enclosed within a mouthpiece and the actuator typically has an orifice diameter of about 0.5 millimeters.

The medicament formulations of the present invention may also be administered utilizing a nebulizer device. Typical commercial nebulizer devices produce dispersions of droplets in gas streams by one of two methods. Jet nebulizers use a compressed air supply to draw liquid up a tube and through an orifice by venturi action and introduce it into a flowing gas stream as droplets suspended therein, after which the fluid is caused to impact one or more stationary baffles to remove excessively large droplets. Ultrasonic nebulizers use an electrically driven transducer to subject a fluid to high-frequency oscillations, producing a cloud of droplets which can be entrained in a moving gas stream; these devices are less preferred for delivering suspensions.

Also available are hand-held nebulizers which atomize a liquid with a squeeze bulb air supply, but the more widely used equipment incorporates an electrically powered compressor or connects to a cylinder of compressed gas. Although the various devices which are commercially available vary considerably in their delivery efficiency for a given medicament since their respective outputs of respirable droplets are far from identical, any may be used for delivery of the medicaments of the present invention when a prescriber specifies an exact amount of medicament formulation which is to be charged to each particular device.

The present invention encompasses also the provision of a medicament comprising a solution containing pleconaril optionally containing one or more other therapeutic agents (described in more detail below, but generally selected depending upon the disease state to be treated), including, but not limited to, corticosteroids, antihistamines, expectorants, non-steroidal anti-inflammatory agents (NSAID agents), decongestants, anti-cholinergics, pharmaceutically acceptable zinc salts, antibiotics, histamine H₃ receptor antagonists, leukotriene D₄ antagonists, leukotriene inhibitors, P₂Y agonists, SYK kinase analogues, 5-lipoxygenase inhibitors, “FLAP antagonists” (defined below), antioxidants, and compounds known for the treatment of the common cold such as echinacea, Vitamin C, Vitamin E and the like. The present invention encompasses also a kit containing at least one medicament comprising a solution containing pleconaril which optionally includes one or more additional therapeutic agents, and optionally includes a wholly separate medicament containing one or more additional therapeutic agents and at least one apparatus for administering the pleconaril-containing medicament. When additional medicaments are included within the kit the apparatus is adapted for simultaneous, sequential, or separate administration of the pleconaril-containing medicament and the separate medicament(s) containing additional therapeutic agent(s).

The above-mentioned additional therapeutic agents may be incorporated into a medicament comprising a solution containing pleconaril by, for example, co-dissolving one or more additional therapeutic agents in a pleconaril-containing solution, suspending one or more additional therapeutic agents having a particulate form in a solution containing pleconaril, dissolving one or more additional therapeutic agents in a solvent miscible with the pleconaril-containing solution and admixing the two solutions, optionally with the inclusion of a cosolvent or surfactant to assist in mixing, dissolving one or more additional therapeutic agents in a solvent which is non-miscible with the pleconaril-containing solution and forming an emulsion between the two solutions, and providing a medicament comprising a solution containing pleconaril and additional therapeutic agents which utilizes two or more of these techniques. When a medicament comprising a solution containing pleconaril is provided with at least one separate medicament comprising one or more additional therapeutic agents, the two or more medicaments may be supplied to an end user in a form that permits simultaneous, sequential, or separate administration of the separate medicaments. Moreover, a solution containing pleconaril and an additional therapeutic agent and one or more other excipients may be administered in combination or separately in the method of treating the disease. For example, they may be administered concurrently or sequentially, i.e. they may be administered in combination either concurrently or by the sequential administration of the constituents of the composition in a suitable order.

An example of a medicament comprising a solution containing pleconaril and one or more additional therapeutic agents is the combination of a solution containing pleconaril of the present invention, for example, pleconaril dissolved in an glyceride oil, admixed with a thixotropic formulation comprising microcrystalline cellulose, an additional therapeutic agent, for example, oxymetazoline hydrochloride, and a polymer selected from an alkali metal carboxyalkylcellulose, a polyvinylpyrrolidone polymer, and mixtures thereof, in the provision of a topical medicament which can be applied to a bodily cavity. An example of one such medicament is a formulation for application to the nasal cavity, via inhalation administration, which, after application, is retained therewithin. Aqueous thixotropic compositions suitable for administration to nasal mucosa are known, for example those described in U.S. Pat. Nos. 6,841,146 (the '146 patent, issued Jan. 11, 2005), 6,824,762 (the '762 patent, issued Nov. 13, 2001), 6,565,832 (the '832 patent, issued May 20, 2003), 6,316,483 (the '484 patent, issued Nov. 13, 2001), and 5,897,858 (the '858 patent, issued Apr. 27, 1999) each to Haslwanter et al., each of which is incorporated herein by reference in its entirety.

In addition to medicaments comprising an emulsion of a pleconaril containing solution and an aqueous thixotropic formulation described above, the present invention encompasses medicaments suitable for administration to the nasal mucosa comprising an aqueous thixotropic formulation and suspended therein, pleconaril particulate material. Such compositions can comprise additionally, one or more additional particulate therapeutic agents co-suspended in the aqueous thixotropic formulation, for example mometasone furoate. U.S. Pat. No. 6,127,353, which is incorporated herein by reference in its entirety, describes a process for suspending mometasone furoate in an aqueous thixotropic formulation suitable for aerosol administration. Surprisingly, micronized pleconaril powder can be employed, utilizing the techniques and excipients described in the '353 patent to provide a medicament comprising an aqueous thixotropic formulation having pleconaril suspended therein which is suitable for administration to the nasal mucosa by dispensing the suspension from a metered dose pump spray device, for example, those described herein.

The inventors have surprisingly discovered that a particulate form of pleconaril having a suitable average particle size and particle size distribution for administration to the nasal mucosa can be suspended in an aqueous thixotropic formulation suitable for administration to the nasal cavity which has “no-drip” properties permitting it to be retained in the nasal cavity after administration. Examples of aqueous formulations having “no-drip” properties include those described in the each of U.S. Pat. Nos. 6,841,146 (the '146 patent), 6,824,762 (the '762 patent), 6,565,832 (the '832 patent), 6,316,483 (the '484 patent), and 5,897,858 (the '858 patent). The formulations and formulating techniques in the '146, '762. '832, '484, and '858 patents are suitable for administration to nasal mucosa utilizing a metered dose pump spray bottle, for example, of the type described above. Accordingly, utilizing the formulating techniques and excipients described in the above-referenced patents along with micronized pleconaril powder provides an aqueous suspension of pleconaril particulate according to the present invention which is suitable for providing an aerosol for administering pleconaril to the nasal mucosa, and, once administered, exhibits “no-drip” properties, permitting the medicament to remain in contact with the mucosa.

A suitable pleconaril suspension in an aqueous thixotropic carrier according to the present invention can be prepared by adding particulate pleconaril in a dispersed form, which contains optionally other therapeutic agents, to a mixture comprising a dispersed gelling agent, for example polyvinylpyrrolidone, as taught in the '858 and '483 patents, or a mixture of microcrystalline cellulose and at least one alkali metal carboxyalkylcellulose, optionally with polyvinylpyrrolidone, as taught in the '146 and '762, and '832 patents, wherein the finished composition contains also one or more members of the group selected from a wetting agent, for example polysorbate 80, preservatives, buffering agents, humectants, flavoring agents, and mixtures of two or more thereof. In general, it is preferred to separately prepare liquid suspensions of each therapeutic agent particulate material, for example, by blending the therapeutic agent particulate material in an aqueous solution of a wetting agent, for example, polysorbate 80, to provide a dispersion of the particulate therapeutic agent, and separately add each such therapeutic agent dispersion to the gelling agent dispersion. Alternatively, a blend of therapeutic agents in a particulate form can be provided from which a dispersion is made in accordance with the above-mentioned procedure, which is then added to the gelling agent dispersion. The teaching of each of the '858, '483, '146, '762, and '832 patents regarding each of the constituents of such suspensions and the techniques for preparing them are incorporated herein by reference.

Particulate materials suitable for application to the nasal mucosa have at least 80% of the particles less than 10 microns, 90% less than 20 microns and not more than 10% greater than 20 microns. A suitable pleconaril particulate can be provided by subjecting the dry powder to standard jet mill miconization.

For embodiments wherein a medicament of the invention comprises suspending an additional therapeutic agent in a solution containing pleconaril, for example, oxymetazoline HCl suspended in a solution containing pleconaril which includes a pleconaril-dissolving glyceride oil solvent, it will be appreciated that the suspension must comprise particles of an appropriate size for the site of administration. For example, medicaments intended for oral inhalation will comprise particles having a respirable size, preferably an average size of less than about 5 microns in the largest dimension and more preferably averaging less than about 2 microns in the largest dimension and have a size distribution of from about 1 to about 5 microns. As will be appreciated, the delivery device utilized to administer the medicament, for example, a nebulizer, a metered pump spray, and a pressurized metered dose inhaler, must provide particle-containing droplets having an appropriate size range for deposition onto the desired area of the respiratory system.

It is believed that the inventive medicaments comprising a solution containing pleconaril, either alone or in combination with other therapeutic agents, will be useful in the treatment of disease states including, but not limited to, asthma, rhinovirus, neonatal sepsis, ALS, type I diabetes, viral induced infections of the upper and lower airways, viral meningitis, and life-threatening diseases such as chronic meningoencephalitis, neonatal enteroviral disease, polio and myocarditis. The compositions of the present invention may be used also prophylactically to prevent exacerbations of symptoms associated with diseases of the upper airways in individuals with such diseases.

The viral based disorders which may be treated by compositions of the present invention include the treatment and/or prevention of the common cold. Compositions of the present invention may be utilized also in preventing exacerbation of disorders of the upper and lower airways. With respect to upper airway disorders, for example, the congestion and nasal blockage associated with allergic rhinitis, sinusitis, fungal induced sinusitis, bacterial based sinusitis, polyposis and the like. Examples with regard to disorders of the lower airways include administration of compositions of the present invention to prevent the need for the use of rescue medications for disorders of the lower airways, for example, asthma, chronic obstructive pulmonary disorder, allergic asthma, and emphysema. The compositions of the present invention may be useful also for the treatment and prevention of the nasal (stuffiness/congestion, rhinorrhea, nasal itching, sneezing) and non-nasal (itchy/burning eyes, tearing/watery eyes, redness of the eyes, itching of the ears/palate) symptoms of seasonal and perennial allergic rhinitis, including nasal congestion, in patients in need of such treatment and/or prevention.

The formulations of the present invention may be used also for post viral-exposure treatment, The compositions may be used also prophylactically, for example, when a household member, for example, a child, is stricken with a cold, or, for example, administered to individuals in settings where there is a high incidence of viral or bacterial based pathogens. Examples of the latter include hospitals, nursing homes, pharmacies and the like.

It is believed that certain of the medicaments of the present invention will have advantages over medicaments which do not comprise a solution containing pleconaril, including but not limited to, administration of pleconaril by inhalation through oral and nasal routes and/or high dose loading availability. It is believed that certain medicaments of the present invention provide also advantages in the provision of pediatric therapy and in facilitating treatment by topical administration of certain medicaments in the provision of therapy for disease states amenable to treatment by those medicaments.

In treatment of disease states responding to pleconaril administration, the medicaments of the present invention are typically utilized in an amount that provides an amount of pleconaril ranging from about 1 mg to about 600 mg, preferably about 200 to about 400 mg in single or divided doses daily for a period sufficient to treat the condition, for example, a viral infection, or more particularly, a viral induced respiratory infection.

The present invention encompasses also ophthalmic compositions containing pleconaril. For ophthalmic compositions, the compositions of the present invention may take various forms. For example, they may be an aqueous gel or liquid, or an ointment. In a preferred embodiment, the composition is a water-in-oil emulsion with the additional therapeutic agent(s) dissolved or suspended within aqueous droplets which are in turn suspended in a lotion or flowable ointment base comprising, e.g., petrolatum, mineral oil, and the like and including pleconaril dissolved in a suitable pleconaril-dissolving glyceride oil or a suitable pleconaril-dissolving HFC. Additional emollient ingredients such as isopropyl myristate may also be added. Such a lotion or ointment covers the conjunctiva and cornea with a thin film that both carries active ingredients and provides for prolonged drainage through the naso-lacrimal ducts. The film also provides a barrier to evaporative loss of water from the corneal stroma.

There follows a list illustrating, but not exhaustively enumerating, examples of the above-mentioned additional therapeutic agents which may be incorporated into a medicament comprising a solution containing pleconaril or administered as a separate medicament along with a medicament comprising a solution containing pleconaril in the treatment of a disease state.

Accordingly, examples of Corticosteroids which may be used in the present invention include, but are not limited to, mometasone furoate, dexamethasone, butoxicort, rofleponide, budesonide, deflazacort, ciclesonide, fluticasone, beclomethasone, loteprednol or triamcinolone. Preferred corticosteroids are fluticasone and mometasone furoate. A particularly preferred corticosteroid is Mometasone Furoate.

Mometasone Furoate is a corticosteroid approved for topical dermatologic use to treat inflammatory and/or pruritic manifestations of corticosteroid-responsive dermatoses. The compound may be prepared in accordance with the procedures disclosed in U.S. Pat. Nos. 4,472,393, 4,731,447, 4,873,335, 5,837,699 and 6,127,353, all of which are hereby incorporated by reference in their entirety. Mometasone Furoate is a topically active steroid which is not readily bioavailable. It is commercially available as a spray for intra-nasal administration under the name of Nasonex®. Mometasone's use for the treatment of airway passages and lung diseases is disclosed in U.S. Pat. Nos. 6,677,323, 6,677,322, 6,365,581, 6,187,765, 6,068,832, 6,057,307 5,889,015 5,837,699 and 5,474,759, all of which are incorporated by reference in their entirety.

Typically, in the treatment of allergic, non-allergic rhinitis and/or inflammatory diseases of the upper or lower airway passages, for example, but not limited to, treatment of asthma, Mometasone Furoate is administered in a substantially non-systemically available form, for example, as a nasal inhalant, in the range of about 10 to 5000 micrograms (“mcg”)/day, 10 to 4000 mcg/day, 10 to 2000 mcg/day, 25-1000 mcg/day, 25 to 400 mcg/day, 25-200 mcg/day, 25-100 mcg/day or 25-50 mcg/day in single or divided doses.

In further example, when the corticosteroid is fluticasone, it may be administered at the dose of 2 sprays of 50 μg of fluticasone propionate each in each nostril once daily. Alternatively, it may be administered at a dose of fluticasone is 1 spray of 50 μg of fluticasone propionate each in each nostril once daily. When the corticosteroid is triamcinolone, it may be administered at a dose of triamcinolone is 220 μg per day as two sprays in each nostril once daily. Alternatively, it may be administered at a dose of 110 μg per day as one spray in each nostril once daily. When the corticosteroid is budesonide, the administered dose of budesonide may be 64 μg per day administered as one spray per nostril of 32 μg once daily.

Examples of Histamine H₁ receptor antagonists (herein also antihistamines) that may be included in or administered in conjunction with a medicament comprising a solution containing pleconaril include, but are not limited to, Astemizole, Azatadine, Azelastine, Acrivastine, Bromphemiramine, Chlorpheniramine, Clemastine, Cyclizine, Carebastine, Cyproheptadine, Carbinoxamine, Desloratadine, Doxylamine, Diphenhydramine, Cetirizine, Dimenhydrinate, Dimethindene, Ebastine, Epinastine, Efletirizine, Fexofenadine, Hydroxyzine, Ketotifen, Loratadine, Levocabastine, Levocetirizine, Mizolastine, Mequitazine, Mianserine, Noberastine, Meclizine, Norastemizole, Picumast, Pyrilamine, Promethazine, Terfenadine, Tripelennamine, Temelastine, Trimeprazine, Triprolidine and mixtures of any two or more of the foregoing. Preferred Histamine H₁ receptors are desloratadine, loratadine, fexofenadine and ceterazine. A medicament comprising a solution containing pleconaril in conjunction with one or more antihistamines (either included in the medicament or provided in a form for simultaneous, sequential or separate administration) may be administered either orally or topically as set forth herein.

Desloratadine is also termed Descarboethoxyloratidine and DCL. DCL is a non-sedating antihistamine, whose technical name is 8-chloro-6,11-dihydro-11-(4-piperidylidene)-5H-benzo[5,6]cyclohepta[1,2]pyridine. This compound is described in Quercia, et al., Hosp. Formul., 28: 137-53 (1993), in U.S. Pat. No. 4,659,716, and in WO 96/20708. The use of Desloratadine for the treatment of congestion is disclosed in U.S. Pat. No. 6,432,972. DCL is an antagonist of the H₁ histamine receptor protein. The H₁ receptors are those that mediate the response antagonized by conventional antihistamines. H₁ receptors are present, for example, in the ileum, the skin, and the bronchial smooth muscle of man and other mammals. The amount of DCL which can be employed in a unit (i.e. single) dosage form of the present compositions can range from about 2.5 to about 45 mg, also from about 2.5 to about 20 mg, also from about 5 to about 10 mg. Preferred dosage amounts include 2.5 mg, 5.0 mg, 10.0 mg and 20.0 mg.

Loratadine is a non-sedating antihistamine whose technical name is 11-(4-piperidylidene)-5H-benzo-[5,6]-cyclohepta-[1,2-b]-pyridine. The compound is described in U.S. Pat. No. 4,282,233. Loratadine is a potent tricyclic and antihistaminic drug of slow release, with a selective antagonist of peripheric H₁ receptors activity.

Fexofenadine reportedly is a non-sedating antihistamine, whose technical name is 4-[1-hydroxy-4-(4-hydroxy-diphenylmethyl)-1-piperidinyl)butyl]-α,α-dimethyl-benzene acetic acid. Preferably the pharmaceutically acceptable salt is the hydrochloride, also known as fexofenadine hydrochloride. The amount of fexofenadine which can be employed in a unit dosage form of the present composition can range from about 40 to 200 mg, also from about 60 to about 180 milligrams, also about 120 milligrams.

Cetirizine hydrochloride reportedly is an H₁ receptor antagonist. The chemical name is (±)-[2-[4-(4-chlorophenyl)phenylmethyl]-1-piperazinyl]ethoxy acetic acid, dihydrochloride. Cetirizine hydrochloride is a racemic compound with an empirical formula of C₂₁H₂₅ClN₂O₃.2HCl. Cetirizine hydrochloride is a white, crystalline powder and is water soluble. Cetirizine hydrochloride is available from Pfizer Inc., New York, N.Y., under the trade name ZYRTEC®. The amount of Cetirizine which can be employed in a unit dosage form of the present composition can range from about 0 to 40 mg, also from about 5 to about 10 milligrams. The levo isomer of Cetirizine may also be combined with Pleconaril in the formulations of the present invention. Another form of Cetirizine for use in the present invention is Cetirizine dinitrate.

Examples of expectorants suitable for use in combination with a medicament comprising a solution of Pleconaril include, but are not limited to, ambroxol, guaiafenesin, terpin hydrate, and potassium quaicolsulfonate. Ambroxol is a bromhexine metabolite, chemically identified as trans-4(2-amino-3,5-dibromobenzil, amine) ciclohexane hydrochloride, which has been widely used during more than two decades as an expectorant agent or stimulating pulmonary surfactant factor. The compound is described in U.S. Pat. No. 3,536,712. Guaiafenesin is an expectorant, whose technical name is 3-(2-methoxyphenoxy)-1,2-propanediol. The compound is described in U.S. Pat. No. 4,390,732. Terpin hydrate is an expectorant, whose technical name is 4-hydroxy-α,α,4-trimethylcyclohexane-methanol. Potassium guaicolsulfonate is an expectorant, whose technical name is 3-Hydroxy-4-methoxybenzenesulfonic acid mix with mono-potassium 4-hydroxy-3-methoxybenzenesulfonate.

Examples of suitable decongestants for use within the scope of the present include both oral and nasal decongestants in combination with Pleconaril. Examples of nasal decongestants useful in the present invention include, without being limited to, the sympathomimetic amine nasal decongestants. Those currently approved for topical use in the United States include, without limitation, levmetamfetamine (also known as 1-desoxyephedrine), ephedrine, ephedrine hydrochloride, ephedrine sulfate, naphazoline hydrochloride, oxymetazoline and pharmaceutically acceptable salts thereof, oxymetazoline hydrochloride, phenylephrine hydrochloride, and propylhexedrine. Oral decongestants for use in the present invention include, without limitation, phenylpropanolamine, phenylephrine and pseudoephedrine as well as pharmaceutically acceptable salts thereof. Pseudoephedrine and its acid additional salts, e.g., those of HCl or H₂SO₄, are recognized by those skilled in the art as a sympathomimetic therapeutic agent that is safe and effective for treating nasal congestion. They are commonly administered orally concomitantly with an antihistamine for treatment of nasal congestion associated with allergic rhinitis. When used in the present invention as a nasal decongestant it is preferred to use pseudoephedrine in amounts of equivalent to about 120 mg pseudoephedrine sulfate dosed one to 4 times daily. However, lesser amounts of pseudoephedrine sulfate may be used in combination with Pleconaril.

Examples of Histamine H₃ receptor antagonists suitable for use in the present invention include, but are not limited to, Thioperamide, Impromidine, Burimamide, Clobenpropit, Impentamine, Mifetidine, S-sopromidine, R-sopromidine, 3-(imidazol-4-yl)-propylguanidine (SKF-91486), 3->(4-chlorophenyl)methyl-5->2-(1H-imidazol-4yl)ethyl 1,2,3-oxadiazole (GR-175737), 4-(1-cyclohexylpentanoyl-4-piperidyl) 1H-imidazole (GT-2016), 2-{>2->4(5)-imidazolylethylthio}-5-nitropyridine (UCL-1199) Clozapine, SCH497079 and SCH539858. Particularly preferred compounds are disclosed and claimed in U.S. Pat. No. 6,720,328 and United States Patent Application Publication No. 20040097483A1, both assigned to Schering Corp., and both of which are hereby incorporated by reference. Other preferred compositions may further include both H₁ and H₃ receptors antagonists as is disclosed in U.S. Pat. No. 5,869,479, also assigned to Schering Corp., which is hereby incorporated by reference. Other compounds can readily be evaluated to determine activity at H₃ receptors by known methods, including the guinea pig brain membrane assay and the guinea pig neuronal ileum contraction assay, both of which are described in U.S. Pat. No. 5,352,707. Another useful assay utilizes rat brain membranes and is described by West et al., “Identification of Two H₃-Histamine Receptor Subtypes,” Molecular Pharmacology, Vol. 38, pages 610-613 (1990).

Examples of Anti-Cholinergic agents for use in the present invention include, but are not limited to, Tiotropium, Oxitropium, Ipratropium, Methantheline, Propantheline, Dicyclomine, Scopolamine, Methscopolamine, Telenzepine, Benztropine, QNX-hemioxalate, Hexahydro-sila-difenidol hydrochloride and Pirenzepine. It is preferred to administer these compositions either orally or nasally as set forth below in amounts that are known to one of skill in the art.

Examples of Antibiotics for use in combination with Pleconaril in the present invention include, but are not limited to macrolides, cephalosporin, and antibacterials. Specific examples of suitable antibiotics include, but are not limited to, Tetracycline, Chlortetracycline, Bacitracin, Neomycin, Polymyxin, Gramicidin, Oxytetracycline, Chloramphenicol, Florfenicol, Gentamycin, Erythromycin, Clarithromycin, Azithromycin, Tulathromycin, Cefuroxime, Ceftibuten, Ceftiofur, Cefadroxil, Amoxicillin, Penicillins, Amoxicillin with clavulanic acid or an other suitable beta-lactamase inhibitor, Sulfonamides, Sulfacetamide, Sulfamethizole, Sulfisoxazole; Nitrofurazone, and Sodium propionate. The therapeutic amounts of compositions which may be administered are known to one of skill in the art.

Examples of P2Y₂ receptor agonists for use in the present invention include, but are not limited, to diquafosol tetrasodium. Diquafosol tetrasodium is a P2Y₂ receptor agonist that activates receptors on the ocular surface and inner lining of the eyelid to stimulate the release of water, salt, mucin and lipids—the key components of natural tears. Mucin is made in specialized cells and acts to lubricate surfaces. Lipids in the eye are oily substances that form the outer-most layer of the tear film and are responsible for the prevention of excess tear fluid evaporation. In preclinical testing, diquafosol reportedly increased the secretions of natural tear components. Diquafosol is available from Inspire. P2Y₂ receptor agonists are a new class of compounds that are being developed for the treatment of a variety of conditions in which mucociliary clearance (MCC) is impaired, including chronic bronchitis and cystic fibrosis (CF). Other mucolytic agents may include N-Acetylcysteine and endogenous ligand compound UTP. These compositions may be administered either orally or nasally as set forth below in amounts that are known to one of skill in the art.

Examples of Non-Steroidal Anti-Inflammatory (“NSAID's”) agents suitable for use with the present invention includes, but is not limited to, Acetylsalicylic acid, Acetaminophen, Indomethacin, Diclofenac, Piroxicam, Tenoxicam, Ibuprofen, Naproxen, Ketoprofen, Nabumetone, Ketorolac, Azapropazone, Mefenamic acid, Tolfenamic acid, Sulindac, Diflunisal, Tiaprofenic acid, Podophyllotoxin derivatives, Acemetacin, Aceclofenac, Droxicam, Oxaprozin, Floctafenine, Phenylbutazone, Proglumetacin, Flurbiprofen, Tolmetin and Fenbufen. These compositions may be administered either orally or nasally as set forth below in amounts that are known to one of skill in the art.

Examples of Leukotriene₄ antagonists and/or inhibitors suitable for use in the present invention include, but are not limited to Zileuton, Docebenone, Piripost, ICI-D2318, MK-591, MK-886, sodium 1-(((R)-(3-(2-(6,7-difluoro-2-quinolinyl)ethynyl)phenyl)-3-(2-(2-hydroxy-2-propyl)phenyl)thio)methyl)cyclopropane-acetate (also referred to herein for convenience as “compound LAcetate”); 1-(((R)-(3-(2-(2,3-dichlorothieno[3,2-b]pyridin-5-yl)-(E)-ethenyl)phenyl)-3-(2-(1-hydroxy-1-methylethyl)phenyl)propyl)thio)-methyl)cyclopropaneacetic acid (also referred to herein for convenience as “compound LAcid”), Pranlukast, Zafirlukast, and Montelukast and the compound [2-[[2-(4-tert-butyl-2-thiazolyl)-5-benzofuranyl]oxymethyl]phenyl]acetic acid (also referred to herein for convenience as “compound FK011” or “FR150011”). Preferred are montelukast, pranlukast, zafirlukast, compounds “FK011”, “LAcetate”, and “LAcid”. Compositions containing these constituents may be administered either orally or nasally as set forth below in amounts that are known to one of skill in the art.

Montelukast is a Leukotriene D₄ antagonist capable of antagonizing the receptors for the cysteinyl leukotrienes. The technical name of Montelukast is [R-(E)]-1-[[[1-[3-[2-(7-chloro-2-quinolinyl)ethenyl]phenyl]-3-[2-(1-hydroxy-1-methylethyl)phenyl]propyl]thio]methyl]-cyclopropaneacetic acid. This compound is described in EP 480,717. A preferred pharmaceutically acceptable salt of Montelukast is the monosodium salt, also known as Montelukast sodium. The amount of Montelukast which can be employed in a unit dosage form of the present invention can range from about one to 100 milligrams, also from about 5 to about 20 milligrams, preferably about 10 milligrams.

The compound 1-(((R)-(3-(2-(6,7-difluoro-2-quinolinyl)ethenyl)phenyl)-3-(2-(2-hydroxy-2-propyl)phenyl)thio)methylcyclopropaneacetic acid is a leukotriene antagonist described in WO 97/28797 and U.S. Pat. No. 5,270,324. A pharmaceutically acceptable salt of this compound is the sodium salt, also known as sodium 1-(((R)-(3-(2-(6,7-difluoro-2-quinolinyl)ethenyl)phenyl)-3-(2-(2-hydroxy-2-propyl) phenyl)thio)-methylcyclopropaneacetate.

The compound 1-(((1(R)-3(3-(2-(2,3-dichlorothieno[3,2-b]pyridin-5-yl)-(E)-ethenyl)phenyl)-3-(2-(1-hydroxy-1-methylethyl)phenyl)propyl)-thio)methyl)cyclopropaneacetic acid is a leukotriene antagonist described in WO 97/28797 and U.S. Pat. No. 5,472,964. A pharmaceutically acceptable salt of this compound is the sodium salt, also known as sodium 1-(((1(R)-3(3-(2-(2,3-dichlorothieno[3,2-b]pyridin-5-yl)-(E)-ethenyl)phenyl)-3-(2-(1-hydroxy-1-methylethyl)phenyl)propyl)-thio)methyl)cyclopropaneacetate.

Pranlukast is a leukotriene antagonist described in WO 97/28797 and EP 173,516. The technical name for this compound is N-[4-oxo-2-(1H-tetrazol-5-yl)-4H-1-benzopyran-8-yl]-p-(4-phenylbutoxy)benzamide. The amount of Pranlukast which can be employed in a unit dosage form can range from about 100 to about 700 mg, preferably from about 112 to about 675 mg; also from about 225 mg to about 450 mg; also from about 225 to about 300 mg.

Zafirlukast is a leukotriene antagonist described in WO 97/28797 and EP 199,543. The technical name for this compound is cyclopentyl-3-[2-methoxy-4-[(o-tolylsulfonyl)carbamoyl]benzyl]-1-methylindole-5-carbamate.

The compound [2-[[2-(4-tert-butyl-2-thiazolyl)-5-benzofuranyl]oxymethyl]phenyl]acetic acid is a leukotriene antagonist and/or inhibitor whose method for preparation is described in U.S. Pat. No. 5,296,495 and Japanese Patent JP 08325265A. An alternative name for this compound is 2-[[[2-[4-(1,1-dimethylethyl)-2-thiazolyl]-5-benzofuranyl]oxy]methyl]-benzeneacetic acid. The code number for this compound is FK011 or FR150011.

Pharmaceutically acceptable zinc salts contemplated for use in the present invention comprise those water soluble salts reported to have beneficial effects against the common cold. Typically such preparations comprise an aqueous or saline solution with a concentration of ionic zinc below that which causes irritation to mucus membranes. Generally the ionic zinc in such solutions is present substantially as unchelated zinc and is in the form of free ionic solution. Zinc ionic solutions for use in the present invention will typically contain substantially unchelated zinc ions in a concentration of from about 0.004 to about 0.12% (w/vol). Preferably the substantially unchelated ionic zinc compound can comprise a mineral acid salt of zinc selected from the group consisting of zinc sulfate, zinc chloride, and zinc acetate. These compositions may be administered either orally or nasally as set forth below in amounts that are known to one of skill in the art.

SYK kinase analogs are a class of molecules which work via a novel mechanism, blocking SYK kinase. Compound R112, available from Rigel Pharmaceuticals, Inc. is an example of an SYK kinase analog. A recent study reportedly showed a greater than 20% relative improvement for R112 over placebo (an absolute difference of 9% over placebo) and up to 38% improvement for R112 from baseline measurements (prior to drug initiation) of symptoms associated with chronic nasal congestion (e.g. stuffy nose) over a placebo.

As used herein, the term “5-lipoxygenase inhibitor” (also referred to as a “5-LO inhibitor”) includes any agent, or compound that inhibits, restrains, retards or otherwise interacts with the enzymatic action of 5-lipoxygenase. Examples of 5-lipoxygenase inhibitors include, but not limited to, zileuton, docebenone, piripost, and the like. As used herein, the associated term “5-lipoxygenase activating protein antagonist” or “FLAP antagonist” includes any agent or compound that inhibits, retrains, retards or otherwise interacts with the action or activity of 5-lipoxygenase activating protein, examples of which include, but not limited, “FLAP antagonists” MK-591 and MK-886.

In addition to those optional therapeutic agents mentioned above which may be incorporated into or used in conjunction with a medicament comprising a pleconaril-containing a solution according to the present invention, when such a medicament is administered to relieve oropharyngeal discomfort, for example, but not limited to, a sore throat, cold or canker sores, and painful gums, the medicament comprising a solution containing pleconaril may include topical anesthetics such as phenol, hexylresorcinol, salicyl alcohol, benzyl alcohol, dyclonine, dibucaine, benzocaine, buticaine, cetylpyridinium chloride, diperidon, clove oil, menthol, camphor, eugenol and others. Medicaments of the invention intended for application to the skin may similarly include a therapeutic agent for relieving skin discomfort including, but not limited to, lidocaine, benzocaine, tetracaine, dibucaine, pramoxine, diphenhydramine, and benzyl alcohol.

As mentioned above, in some embodiments the medicaments of the invention comprising a solution containing pleconaril can also be incorporated into any other dosage form suitable for incorporation of a liquid. For example, as will be appreciated, medicaments comprising a solution containing pleconaril of the invention may be provided in a form suitable for administration by ingestion, for example, but not limited to, a syringe-dispensed liquid for pediatric use and incorporation of a solution containing pleconaril into a gelatin capsule. It is preferred to administer a medicament comprising a solution containing pleconaril as set forth herein in a manner in which the medicament is substantially non-systematically bioavailable.

For oral dosage form preparations, a pharmaceutically acceptable carrier (which includes diluents, excipients or carrier materials) is also present in the composition. The carrier is suitably selected with respect to the intended form of administration, i.e. oral tablets, capsules (either solid-filled, semi-solid filled or liquid filled), powders for constitution, oral gels, elixirs, syrups, suspensions, and the like, and consistent with conventional pharmaceutical practices. For example, for oral administration in the form of tablets or capsules, the active drug component may be combined with any oral non-toxic pharmaceutically acceptable inert carrier, such as lactose, starch, sucrose, cellulose, magnesium stearate, dicalcium phosphate, calcium sulfate, mannitol, ethyl alcohol (liquid forms) and the like. Moreover, when desired or needed, suitable binders, lubricants, disintegrating agents, disinfectants and coloring agents may also be incorporated in the mixture. Suitable binders include starch, gelatin, natural sugars, corn sweeteners, natural and synthetic gums such as acacia, sodium alginate, carboxymethylcellulose, polyethylene glycol and waxes. Among the lubricants there may be mentioned for use in these dosage forms, boric acid, sodium benzoate, sodium acetate, sodium chloride, and the like. Disintegrants include starch, methylcellulose, guar gum and the like. Disinfectants include benzalkonium chloride and the like. Sweetening and flavoring agents and preservatives may also be included where appropriate.

The following non-limiting examples illustrate the invention.

Unless otherwise noted, all materials were API or USP grade.

### Example 1

### MDI Dispenser Containing Pleconaril Dissolved in 1,1,1,2,3,3,3 Tetrafluoroethane

Into a standard aluminum 10 ml aerosol canister (source) was placed approximately 150 mg of pleconaril API grade obtained from Viropharma. A 50 microliter dosing valve was crimped onto the canister using a Pamasol Autoguard Crimper®. The canister was charged with 10 g of 1,1,1,2,3,3,3 heptafluoropropane (HFA 227), obtained from Solvay Fluor.

Two additional 10 ml canisters containing 150 mg of pleconaril and 10 g of HFC 227 were prepared using the same method. These canisters were evaluated for stability at room temperature (about 25° C.). Initially each of the canisters delivered about 95% of the expected amount of pleconaril based on the amount charged into the canister and the volume of solution delivered by the dosing valve (about 50 microliters). As shown in Table 1, after one month of inverted storage each canister was found to deliver at least 98% of the same amount of pleconaril initially delivered. After 3 months of inverted storage at 40 C, 75% RH, two canisters were found to deliver at least 98% of the same amount of pleconaril initially delivered and one delivered at least 96% of the same amount of pleconaril initially delivered. This demonstrates that the pleconaril. HFA solutions of the invention are stable.

TABLE 1
Pleconaril Solution MDI Stability Results

| Storage Condition | Can # | % Recovery |
|---|---|---|
| Initial | 1 | 93.67 |
| | 2 | 98.40 |
| | 3 | 96.33 |
| Inverted | 1 | 93.13 |
| 1 month/40 C. | 2 | 96.60 |
| 75% RH | 3 | 95.47 |
| Inverted | 1 | 93.56 |
| 3 month/40 C. | 2 | 95.02 |
| 75% RH | 3 | 95.08 |

Pleconaril containing MDI cans were evaluated for particle size distribution and content uniformity at 6 months after being stored at 40 C and 75% RH as shown in Tables 2, 3 and 4. Cans are primed by actuating the can four times prior to testing. The content uniformity testing is conducted with the first two actuations after priming. Results showed good particle size distribution and content uniformity.

TABLE 2
Particle Size Distribution for Pleconaril Solution Aerosol MDI

| | Micrograms Drug Recovered | | | | |
|---|---|---|---|---|---|
| Particle Size (microns) | Can 1 | Can 2 | Can 3 | AVG | STDEV |
| Entry Port (USP) | 866.41 | 845.71 | 831.29 | 847.8 | 17.7 |
| 10 | 21.78 | 21.71 | 21.41 | 21.6 | 0.2 |
| 9 | 66.54 | 65.40 | 68.95 | 67.0 | 1.8 |
| 5.8 | 108.48 | 116.32 | 102.97 | 109.3 | 6.7 |
| 4.7 | 249.05 | 267.21 | 274.35 | 263.5 | 13.0 |
| 3.3 | 281.41 | 288.22 | 301.78 | 290.5 | 10.4 |
| 2.1 | 224.42 | 247.19 | 228.18 | 233.3 | 12.2 |
| 1.1 | 60.98 | 66.02 | 66.63 | 64.5 | 3.1 |
| 0.65 | 25.31 | 26.01 | 26.93 | 26.1 | 0.8 |
| 0.43 | 21.78 | 18.59 | 24.59 | 21.7 | 3.0 |
| Stage Casings | 39.57 | 31.01 | 35.87 | 35.5 | 4.3 |
| Total | 1965.73 | 1993.41 | 1982.96 | 1980.7 | 14.0 |
| Fine Particles | 862.95 | 913.25 | 922.46 | 899.6 | 32.0 |
| % Fine Particles | 43.90 | 45.81 | 46.52 | 45.4 | 1.4 |
| % Total Recovered | 93.07 | 94.38 | 93.89 | 93.8 | 0.7 |
| Avg Shot wt (mg) | 70.59 | 70.52 | 70.57 | 70.6 | 0.0 |

Time Point: 6 Months
Condition: 40 C. 75% RH
Method: Andersen Impaction
Pleconaril Theoretical Amount: 1.056 mg/actuation

TABLE 3
Particle Size Distribution for Pleconaril
Solution Aerosol MDI % Drug Recovered

| | % Drug Recovered | | | | |
|---|---|---|---|---|---|
| Particle Size (microns) | 1 | 2 | 3 | AVG | STD DEV |
| Entry Port | 44.08 | 42.43 | 41.92 | 42.8 | 1.1 |
| 10 | 1.11 | 1.09 | 1.08 | 1.1 | 0 |
| 9 | 3.39 | 3.28 | 3.48 | 3.4 | 0.1 |
| 5.8 | 5.52 | 5.84 | 5.19 | 5.5 | 0.3 |
| 4.7 | 12.67 | 13.4 | 13.84 | 13.3 | 0.6 |
| 3.3 | 14.32 | 14.46 | 15.22 | 14.7 | 0.5 |
| 2.1 | 11.42 | 12.4 | 11.51 | 11.8 | 0.5 |
| 1.1 | 3.1 | 3.31 | 3.36 | 3.3 | 0.1 |
| 0.65 | 1.29 | 1.3 | 1.36 | 1.3 | 0 |
| 0.43 | 1.11 | 0.93 | 1.24 | 1.1 | 0.2 |
| Stage Casings | 2.01 | 1.56 | 1.81 | 1.8 | 0.2 |
| Total | 100 | 100 | 100 | 100 | 0 |
| % Label Claim | 53.91 | 56.02 | 56.27 | 55.4 | 1.3 |
| MMAD | 2.39 | 2.4 | 2.36 | 2.38 | 0.02 |
| GSD | 2.09 | 2.05 | 2.08 | 2.07 | 0.02 |

Time Point: 6 Months
Condition: 40 C. 75% RH
Method: Andersen Impaction
Pleconaril Theoretical Amount: 1.056 mg/actuation

TABLE 4
Content Uniformity Data for Pleconaril Solution Aerosol MDI

| | | Pleconaril Delivery | | |
|---|---|---|---|---|
| Storage | | | % Label | Shot Weight |
| Condition | Can # | mg/actuation | Claim | mg/actuation |
| Initial | 1 | 1.012 | 95.8 | 70.9 |
| | 2 | 1.000 | 94.7 | 69.8 |
| | 3 | 1.004 | 95.1 | 70.8 |
| | Average | 1.005 | 95.2 | 70.5 |
| | % RSD | 0.6 | 0.6 | 0.9 |
| 1 month/40 C. | 1 | 1.006 | 95.2 | 72.4 |
| 75% RH | 2 | 0.980 | 92.8 | 70.3 |
| | 3 | 0.971 | 91.9 | 69.5 |
| | Average | 0.986 | 93.3 | 70.7 |
| | % RSD | 1.8 | 1.8 | 2.1 |
| 3 months/40 C. | 1 | 1.001 | 94.8 | 71.1 |
| 75% RH | 2 | 1.040 | 98.5 | 70.2 |
| | 3 | 0.998 | 94.5 | 71.5 |
| | Average | 1.013 | 95.9 | 70.9 |
| | % RSD | 2.3 | 2.3 | 0.9 |
| 6 months/40 C. | 1 | 0.965 | 91.3 | 70.0 |
| 75% RH | 2 | 0.971 | 91.9 | 70.7 |
| | 3 | 0.976 | 92.4 | 71.2 |
| | Average | 0.970 | 91.9 | 70.6 |
| | % RSD | 0.6 | 0.6 | 0.9 |

### Example 2

### Nasal Spray Compositions Containing Pleconaril

Nasal spray compositions containing pleconaril were prepared in accordance with the following procedure. Into a vessel was placed 5 kg of purified water. With stirring, 200 g of Avicel RC-591® (mixture of microcrystalline cellulose and sodium carboxymethyl cellulose, obtained from FMC, used as received) was dispersed in the water, following which, 200 g of glycerin was added. In a separate vessel containing 400 g of purified water, 20 g of citric acid (USP article of commerce, used as received) and 28 g of sodium citrate (USP article of commerce, used as received) were dissolved to form a citrate buffer solution. The citrate buffer solution was added to the prepared Avicel/glycerin dispersion.

In a separate vessel containing 2.5 Kg of purified water, 4.0 g of the disodium salt of ethylene-diamine-teteracetic acid (Di-sodium EDTA, USP grade, article of commerce, used as received) were dissolved with stirring. In a separate vessel 1.0 g of Polysorbate 80 (trade name for article of commerce comprising copolymer product of 20 moles of ethylene oxide with 1 mole each of oleate ester of sorbitol and its anhydride, used as received) was dissolved in 200 g of purified water with stirring. This Polysorbate 80 solution was added to the sodium EDTA solution. With continued stirring, 25 g of benzyl alcohol and 150 g of pleconaril (API micronized powder obtained from Viropharma) were dispersed in the Polysorbate 80/sodium EDTA solution. The Polysorbate 80/pleconaril dispersion was added to the Avicel/glycerin/buffer mixture with continued stirring. With continued stirring, an amount of a 50% benzalkonium chloride solution equivalent to 2 g of benzylalkonium chloride was dissolved into the Polysorbate 80/pleconaril dispersion. Purified water was added to bring the mixture to 10 Kg. This mixture provides a formulation containing 15 mg/g of pleconaril, 0.1 mg/g of polysorbate 80, 20 mg/g of Avicel RC-591, 20 mg/g of glycerin, 2.0 mg/g of citric acid, 2.8 mg/g of sodium citrate 0.2 mg/g of benzalkonium chloride, 2.5 mg/g of benzyl alcohol and 0.4 mg/g of EDTA.

Using the same procedure, compositions for suitable for use as a nasal spray were prepared using the constituents, in the amounts indicated, in Table I below. Constituents not previously identified are USP or pharmaceutical grade and are generally identified, where possible, by adopted names, such as are given in the International Cosmetic Ingredient Dictionary and Handbook, 7^{th} edition, J. A. Wenninger et al. Eds., The Cosmetic, Toiletry and Fragrance Association, Washington, D.C., U.S.A. 1997.

| | Weight of indicated constituent expressed as (mg) constituent/(g) of composition | | | | | | | | |
|---|---|---|---|---|---|---|---|---|---|
| Constituent | Exp 1A | Exp 1B | Exp 1C | Exp 1D | Exp 1E | Exp 1F | Exp 1G | Exp 1H | Exp 1I |
| Pleconaril | 15.0 | 15.0 | 15.0 | 15.0 | 15.0 | 15.0 | 15.0 | 15.0 | 15.0 |
| Avicel* | 20.0 | 20.0 | 20.0 | 20.0 | 20.0 | 20.0 | 20.0 | 20.0 | 20.0 |
| Citric Acid | 2.0 | 2.0 | 2.0 | 2.0 | 2.0 | 2.0 | 2.0 | 2.0 | 2.0 |
| Sodium Citrate | 2.8 | 2.8 | 2.8 | 2.8 | 2.8 | 2.8 | 2.8 | 2.8 | 2.8 |
| Di-sodium | 0.4 | 0.4 | 0.4 | 0.4 | 0.4 | 0.4 | 0.4 | 0.4 | 0.4 |
| EDTA | | | | | | | | | |
| Proplyene | 20.0 | 20.0 | — | — | — | — | — | — | — |
| Glycol | | | | | | | | | |
| Glycerin | — | — | 20.0 | 20.0 | 20.0 | 20.0 | 20.0 | 20.0 | 20.0 |
| Methyl Paraben | 1.8 | 1.8 | — | — | — | — | — | — | — |
| Propyl Paraben | 0.2 | 0.2 | — | — | — | — | — | — | — |
| Benzalkonium | — | 0.2 | 0.2 | 0.2 | 0.2 | 0.2 | 0.2 | 0.2 | 0.2 |
| Chloride | | | | | | | | | |
| Phenyl Ethyl | — | — | — | 2.5 | — | — | — | — | — |
| Alcohol | | | | | | | | | |
| Benzyl Alcohol | — | — | 2.5 | — | 2.5 | 2.5 | 2.5 | 2.5 | 2.5 |
| Poloxamer 407 | 0.1 | 0.1 | 0.1 | — | — | — | 0.1 | — | — |
| Tween-80 | — | — | — | 0.1 | 0.1 | 0.1 | — | 0.1 | 0.1 |

| | |
|---|---|
| Water | qs as needed to provide a 1 g sample. |

When aliquots of each of the compositions of Examples 1 to 11 were placed into a metered dose pump spray dispenser equipped with a Valois VP3/93 crimp-on pump they were found suitable for use as a nasal spray composition. Each of these compositions were subjected to stability study at elevated temperature (greater than 40° C.) and were found to be stable for at least three months.

### Example 3

### Thixotropic Nasal Spray Compositions Containing Pleconaril

Pleconaril-containing thixotropic nasal spray compositions according to the present invention are prepared by the following procedure. Into a vessel is placed 725 g of purified water. With stirring, 30 g of Avicel RC-591 is dispersed in the water, and high-shear mixing is applied to the dispersion to insure that the Avicel is dispersed. In a separate vessel containing about 85 g of water, 30 g of Providone is dissolved and stirred until a clear solution is obtained. To the Providone solution, 50 g of PEG-32 (Carbowax™ PEG 1450 from Union Carbide) is added with stirring until a clear solution is obtained. The Providone/PEG-32 solution is added to the Avicel dispersion with continued stirring. In a separate vessel containing about 12 ml of purified water, 0.3 g of Disodium EDTA is added with stirring. When the disodium EDTA is dissolved, 0.95 g of dibasic sodium phosphate and 5.39 g of monobasic sodium phosphate is added to the EDTA solution forming a phosphate buffer solution. The phosphate buffer solution is added to the Avicel dispersion with continued stirring. In a separate vessel, 1.2 g of Polysorbate 80 is dissolved in 400 ml of purified water with stirring. Into the polysorbate 80 solution is dispersed 150 g of pleconaril micronized powder with high shear mixing. The polysorbate 80/pleconaril dispersion is added to the Avicel dispersion with continued stirring. To the pleconaril/Avicel dispersion, 2.5 g of benzalkonium chloride and 3.0 g of benzyl alcohol is added and stirred until dissolved. With continued stirring, purified water is added to the mixture to provide a mixture weight of 1 kg. The mixture is then subjected to high shear mixing to insure that any coagulated particles are redispersed.

When this mixture is placed into a metered dose pump spray dispenser equipped with a Valois VP3/93 crimp-on pump it should be suitable for use as a nasal spray in the provision of pleconaril to nasal mucosa. It is believed that it will also be found to have “no-drip” properties when administered to nasal mucosa.

### Example 4

### Medicament Containing a Pleconaril Solution Comprising Miglyol 812®

Into a vessel is placed 100 ml Miglyol 812® (a triglyceride made from a mixture of saturated fatty acids comprising from about 50 wt. % to about 65 wt. % C₈ and from about 30 wt. % to about 45 wt. % C₁₀ from Sasol North America Inc., USP grade used as received). Into the triglyceride oil, 4 g of micronized pleconaril (API grade, Viropharma) is placed with stirring until the pleconaril is dissolved and a clear solution is provided. It is believed that when this solution is placed in a metered dose pump spray bottle it can be dispensed as an aerosol suitable for inhalation administration of pleconaril to nasal mucosa.

1. A medicament comprising a solution containing pleconaril or a pharmaceutically acceptable salt thereof, wherein at least one solvent comprising said solution is a pleconaril-dissolving hydrofluorocarbon.

2. The medicament of claim 1 wherein said pleconaril-dissolving hydrofluorocarbon is at least one member of the group consisting of 1,1,1,2,3,3,3 heptafluoropropane and 1,1,1,2 tetrafluoroethane.

3. The medicament of claim 2 wherein said pleconaril-dissolving hydrofluorocarbon is 1,1,1,2,3,3,3 heptafluoropropane.

4. The medicament of claim 1 further comprising at least one corticosteroid selected from the group consisting of mometasone furoate, dexamethasone, butoxicart, rofleponide, budesonide, deflazacort, ciclesonide, fluticasone, beclomethasone, leteprednol, and triamcinolone.

5. The medicament of claim 1 further comprising mometasone furoate.

6. The medicament of claim 1 further comprising at least one decongestant selected from the group consisting of pseudoephedrine, phenylpropanolamine, levmetamfetamine, ephedrine, ephedrine hydrochloride, ephedrine sulfate, naphazoline hydrochloride, oxymetazoline or a pharmaceutically acceptable salt thereof, phenylephrine hydrochloride, propylhexedrine, and xylometazoline hydrochloride.

7. The medicament of claim 6 wherein the decongestant is oxymetazoline or oxymetazoline hydrochloride.

8. The medicament of claim 7 wherein oxymetazoline or a pharmaceutically acceptable salt thereof is present as a suspension in said solution containing pleconaril.

9. The medicament of claim 1 further comprising one or more members of the group consisting of corticosteroids, antihistamines, expectorants, non-steroidal anti-inflammatory agents, decongestants, anti-cholinergics, pharmaceutically acceptable zinc salts, antibiotics, histamine H₃ receptor antagonists, leukotriene D₄ antagonists, leukotriene inhibitors, P₂Y agonists, syk kinase analogues, echinaceia, vitamin C, and vitamin E.

10. The medicament of claim 1 further comprising at least one anti-histamine selected from the group consisting of astemizole, azatadine, azelastine, acrivastine, bromphemiramine, cetirizine, chlorpheniramine, clemastine, cyclizine, carebastine, cyproheptadine, carbinoxamine, desloratadine, doxylamine, diphenhydramine, epinastine, efletirizine, fexofenadine, hydroxyzine, ketotifen, loratadine, levocabastine, levocetirizine, mizolastine, mequitazine, mianserine, noberastine, meclizine, norastemizole, picumast, pyrilamine, promethazine, terfenadine, tripelennamine, temelastine, trimeprazine, triprolidine, and mixtures of two or more thereof.

11. The medicament of claim 1 further comprising at least one expectorant selected from the group consisting of ambroxol, guaiafenesin, terpin hydrate, potassium guaicolsulfonate, and carbocistiene.

12. The medicament of claim 1 further comprising at least one non-steroidal anti-inflammatory agent selected from the group consisting of acetyl salicylic acid, acetaminophen, indomethacin, diclofenac, piroxicam, tenoxicam, ibuprofen, naproxen, ketoprofen, nabumetone, ketorolac, azapropazone, mefenamic acid, tolfenamic acid, sulindac, diflunisal, tiaprofenic acid, podophyllotoxin derivatives, acemetacin, aceclofenac, droxicam, oxaprozin, floctafenine, phenylbutazone, proglumetacin, flurbiprofen, tolmetin, and fenbufen.

13. The medicament of claim 1 further comprising at least one anti-cholinergic selected from the group consisting of tiotropium, oxitropium, ipratropium, methantheline, propantheline, dicyclomine, scopolamine, methylscopolamine, telenzepine, benztropine, QNX-hemioxalate, hexahydro-siladifenidol hydrochloride, and pirenzepine.

14. The medicament of claim 1 further comprising at least one antibiotic selected from the group consisting of antibacterials, macrolides and cephalosporins.

15. The medicament of claim 14 wherein the antibiotic is selected from the group consisting of tetracycline, chlortetracycline, bacitracin, neomycin, polymyxin, gramicidin, oxytetracycline, chloramphenicol, flofenicol, gentamycin, erythoromycin, clarithromycin, azithromycin, tulathromycincefurpxo,e. ceftobitem. ceftiofur, defadroxil, amoxicillin, penicillin, amoxicillin combined with a beta-lactamase inhibitor, sulfonamides, sulfacetamide, sulfamethizole, sulfisoxazole, nitrofurazone, and sodium propionate.

16. An aerosol inhalation dosage form comprising the medicament of claim 1.

17. The aerosol dosage form of claim 16 comprising a device providing an aerosol for nasal inhalation.

18. The aerosol dosage form of claim 16 comprising a device providing an aerosol for oral inhalation.

19. An aerosol inhalation dosage form comprising a first medicament of claim 1 wherein said first medicament is packaged for simultaneous, sequential, or separate inhalation administration of at least one additional medicament comprising a solution or suspension containing at least one member of the group consisting corticosteroids, antihistamines, expectorants, non-steroidal anti-inflammatory agents, decongestants, anti-cholinergics, pharmaceutically acceptable zinc salts, antibiotics, histamine H₃ receptor antagonists, leukotriene D₄ antagonists, leukotriene inhibitors, P₂Y agonists, syk kinase analogues, echinaceia, vitamin C, vitamin E and combinations of two or more thereof.

20. A method of treatment of an upper or lower respiratory, viral, inflammatory, or obstructive airway disease comprising administration of an effective amount of a medicament of claim 1.

21. The method of claim 20 wherein administration is carried out using an aerosol dosage form.

22. A pharmaceutical kit comprising at least one medicament of claim 1 together with at least one inhalation device for administering said medicament.

23. A method of providing an aerosol dosage form comprising the medicament of claim 1, the method comprising charging into an aerosol canister fitted with a selected metered dosing valve and containing a weighed amount of pleconaril or a pharmaceutically acceptable salt thereof, a weight of a pleconaril-dissolving hydrofluorocarbon calculated to provide a desired solution concentration of a pleconaril or pharmaceutically acceptable salt thereof.

24. A method of providing an aerosol dosage form delivering pleconaril or a pharmaceutically acceptable salt thereof, the method comprising charging into an aerosol canister fitted with a selected metered dosing valve a weighed amount of a solution comprising pleconaril or a pharmaceutically acceptable salt thereof, dissolved in a pleconaril-dissolving hydrofluorocarbon, and optionally adding a propellant thereto wherein the aerosol dosage form is provided by actuation of said metering valve.

25. The method of claim 24 wherein the solvent is a hydrofluorocarbon selected from 1,1,1,2,3,3,3 heptafluoropropane, 1,1,1,2 tetrafluoroethane and mixtures thereof.
